(19) United States
(12) Patent Application Publication
(10) Pub. No.: US 2009/0215885 A1
(43) Pub. Date: Aug. 27, 2009

(54) Antioxidant Food Composition and Methods and Uses Thereof

(75) Inventors: Paul E. Bendheim, Paradise Valley, AZ (US); Edward Wein, Toronto (CA)

Correspondence Address: PERKINS COIE LLP, POST OFFICE BOX 1208, SEATTLE, WA, 98111-1208, US

(73) Assignee: BRAINSAVERS LLC, Scottsdale, AZ (US)

(21) Appl. No.: 11/920,072

(22) PCT Filed: May 9, 2006
(86) PCT No.: PCT/US2006/017646

§ 371 Date: May 13, 2009

Related U.S. Application Data
(60) Provisional application No. 60/679,335, filed on May 10, 2005.

(51) Int. Cl. A61K 31/355; A23L 1/302

(52) U.S. Cl. 514/458; 426/72

(57) ABSTRACT

Various food compositions are provided that provide nutritional components useful, only or in combination with a training regime, to prevent or delay the onset of cognitive decline.

### TECHNICAL FIELD

The present invention provides various food compositions that are useful for delaying or preventing cognitive decline. The food composition may be used alone or in combination with a physical fitness program and/or a cognitive training program. In one embodiment, the food composition is a bar and comprises at least one antioxidant in its natural matrix and at least one selected ingredient in its processed form.

### BACKGROUND

### I. Cognitive Decline

Cognitive decline may be age-related or disease-related or both. The symptoms and signs are generally similar regardless of etiology and include deterioration in memory, learning ability, attention, concentration, verbal skills, speed of cognitive processing and a variety of other cognitive functions.

Age is a major risk factor for neurodegenerative diseases, for example Alzheimer's, Parkinson's and Pick's Diseases, and stroke and vascular dementia. As society ages, neurodegenerative diseases are becoming increasingly common. Worldwide, the population of people 60 years and older is expected to double in the next 50 years and the elderly will outnumber the young.

Aging is associated with mitochondrial dysfunction, which triggers release of reactive oxygen species (ROS) and subsequent induction of peroxidative reactions that result in cell death and tissue damage. These pathological events are involved in neurodegenerative, cardiovascular, carcinogenic and other age-related tissue specific processes. For example, free radicals may induce neuronal cell dysfunction and death with increasing brain tissue loss over time. Clinically this may be manifested as cognitive decline and a specific neurodegenerative disease such as Alzheimer's disease with its devastating memory loss.

Currently, there are no cures and few treatments for treating cognitive decline and neurodegenerative diseases in general and methods for the prevention or delay of disease onset and progression are being actively sought.

### II. Nutrition and Brain Function

The vitamins, minerals, fatty acids and a host of other biological molecules and micronutrients contained in natural foods and nutritional, food and diet supplements affect brain function in several ways. Ingested nutrients may be used as components in the synthesis of simple and complex molecules (carbohydrates, lipids, and proteins) critical for neuronal, glial, and vascular cell structure and function within the brain, and in the synthesis of neurotransmitters that in part enable normal cognitive function. Certain nutrients, especially some vitamins, micronutrients and minerals have also been shown to be critical in the maintenance of healthy brain cells.

There is accumulating scientific data showing that protection from certain chronic diseases associated with aging involves antioxidant activities, mitochondrial stabilizing/protective activities, metal chelating activities and inhibition of apoptosis of vital cells, and other proposed disease-protecting mechanisms. Numerous studies have demonstrated the health benefits of anti-oxidants and in particular, anti-oxidants derived from natural sources in the adult population. Specifically, vitamin E from natural diet sources has been shown to dramatically reduce the risk of Alzheimer's disease. In particular, two prospective epidemiological studies, one based in the U.S. (Chicago Health and Aging Project (CHAP)) and the other in Holland (the Rotterdam Study), summarized the benefit of consuming dietary natural vitamin E rather than supplements. In the first of these two, a prospective study of individuals selected in a stratified random sample of community-dwelling residents suggested that vitamin E from food, but not from supplements and not other anti-oxidants, is associated with a reduced risk of AD. (Morris et al., 2002). The Rotterdam Study, a population-based, prospective cohort study conducted in the Netherlands concluded that a high dietary intake of vitamin C and vitamin E may lower the risk of Alzheimer disease (Englehart et al., 2002).

Additionally, a host of scientific studies document the benefits of vitamin E and other natural anti-oxidants (vitamin C, beta-carotene, etc.) in cancer prevention, reduction of risk from cardio-vascular disease, and other medical/health benefits.

The literature however has also provided some non-supportive, inconclusive and ambiguous data regarding the benefits of certain antioxidants in the prevention of age-related or disease-related dementia.

Intakes of beta-carotene, flavonoids, and vitamins E and C were shown to be non-correlative with the risk of dementia or its subtypes in the Honolulu-Asia Aging Study (HAAS). The HAAS study suggested that midlife dietary intake of antioxidants does not modify the risk of late-life dementia or its most prevalent subtypes (Laurin et al., 2004).

In yet another study, the relationship between AD and the intake of carotenes, vitamin C, and vitamin E was determined in 980 elderly subjects who were free of dementia at baseline and were followed for a mean time of 4 years. Their conclusion: neither dietary, nor supplemental, nor total intake of carotenes and vitamins C and E was associated with a decreased risk of AD (Luchsinger et al., 2003).

Importantly, there are no reports of health risks of natural vitamins taken in reasonable doses. This is not true of supplemental, non-natural vitamins. Most recently, high-dose supplemental vitamin E has been linked to an increase risk of certain cardiovascular adverse events.

Certain dietary compositions are known in the art. For example, U.S. Pat. No. 6,140,304 discloses a nutritional composition useful for reducing hyperinsulinemia compromising about 20-40% protein (total caloric value), about 30-50% carbohydrates wherein the carbohydrate source has a low glycemic index, a fat source of about 20-40% comprising a combination of activated omega 3 and omega 6 fatty acids in a ratio greater than 2:1.

U.S. Pat. No. 4,451,488 discloses a shelf stable food bar having a soft and chewy texture resulting from a combination of polyhydric alcohols in the mixture. The food bar contains, inter alia, an antioxidant, which is included in a trace amount.

U.S. Pat. No. 5,834,044 discloses a method of preparing a health food product comprising two components, wherein the first component is in the form of a discrete portion from the second component and comprises an antioxidant or a blend of antioxidants. The food product provides enhanced oxidant defense indices and reduces oxidant stress and damage resulting from intense exercise. The product comprises about 0.01% to about 4% (w/w) antioxidants of the total product.

U.S. Pat. No. 5,985,936 discloses a method of preventing and delaying onset of Alzheimer's disease comprising administering to the animal a phytosterol composition, which comprises beta-sitosterol, campesterol and stigmastanol. The '936 patent teaches that the plant sterols may have anti-oxidant activity, thereby protecting the brain from oxidative stress leading to a delay in onset or even prevention of Alzheimer's disease.

U.S. Pat. No. 6,579,544 discloses a dietary supplement in the form of a capsule containing vitamins, minerals, and carotenoids and also preferably bioflavonoids, omega-3 fatty acids, cartilage protectors, and other nutrients beneficial for promoting good health and preventing disease.

U.S. Pat. No. 6,426,362 discloses a nutritional supplement comprising non-natural occurring compounds to ameliorate stress responses in cells. The compositions comprise a tocopherol and a synergist selected from lactoferrin, a flavonoid and a combination of flavonoids. Compounds in their natural matrix are not addressed in that patent.

U.S. Pat. No. 6,914,071 discloses a diet and method to inhibit the onset of deterioration of the mental capacity of a companion pet. The diet comprises an antioxidant or mixture of antioxidants. The application neither teaches nor suggests a composition useful for administration to a human subject.

### REFERENCES

The following references and any additional references cited in the text are hereby incorporated by reference.

Bienias, J. L. et al. (2003) J. Alzheimers Dis. 5:349-55.

Christen, S. et al. (1997) Proc. Nat'l. Acad. Sci., U.S.A. 94:3217-22.

Emerson Lombardo, N. B. et al. Alzheimers Association International Conference on Prevention of Dementia (June 18-21, 2005).

Engelhart, M. J. et al. (2002) JAMA 287:3223-9.

Ferrari, C. K. (2004) Biogerontology 5:275-89.

Giselle, P. L. et al. (2005) J. Neuroscience 25:3032-40.

Laurin, D. et al. (2004) Am. J. Epidemiol. 159:959-6.

Luchsinger, J. A. et al. (2003) Arch. Neurol. 60:203-8.

Meydani and Mosen (1995) Lancet345:170-75.

Milgram, N. W. (2005) Neurobiol. Aging 26:77-90.

Morris, M. C. et al. (2002) JAMA 287:3230-7

Morris, M. C et al. (2002) Arch. Neurol. 59:1125-32

Morris, M. C. et al. (2005) Arch. Neurol. 62:1849-53

Morris, M. C. et al. (2005) Arch. Neurol. 62:641-45 Petersen, R. C. et al. (2005) N. Engl. J. Med. 352:2379-88

### SUMMARY

The present invention provides a nutritional product that provides a dose of at least one antioxidant in its natural matrix and having high bioavailability. The present invention also provides a consumable, flavorful food product that provides dietary nutrients that may be combined with either one or both of a physical fitness plan and cognitive training useful for preventing cognitive decline and preserving and enhancing cognitive function in humans.

In one aspect, a food composition is provided, comprising:

a) vitamin E in its natural matrix, in an amount of from about 1.4 to about 10.5 mg equivalents alpha tocopherol per serving;

b) a complex phyto-antioxidant in its natural matrix; and

c) at least one processed ingredient present in its synthetic, isolated or extract form and selected from the group consisting of vitamin B12, vitamin B6, selenium, folic acid, alpha lipoic acid, ginkgo biloba, co-enzyme Q10, acetyl carnitine, phosphatidyl serine, vinpocetine, DHA, epigallocatechin-3-gallate (EGCG), phosphatidyl serine, indole-3-propionic acid, resveratrol, and salicin from white willow bark. In particular embodiments, one or more of the processed ingredients is excluded from the food composition.

In some embodiments, the complex phyto-antioxidant comprises at least one of an anthocyanin polyphenol, a proanthocyanin polyphenol, a flavanol polyphenol, a flavonol polyphenol, a carotenoid, a terpene and a lignan.

In some embodiments, the complex phyto-antioxidant has a total antioxidant capacity (TAC) of 250 to 5000 micromoles Trolox equivalents (TE) per serving. In particular embodiments, the complex phyto-antioxidant has a TAC of 500 to 5000 micromoles TE per serving.

In some embodiments, the complex phyto-antioxidant has a total phenolic content (TP) of 60 to 1200 mg Gallic Acid Equivalents (GAE) per serving. In particular embodiments, the complex phyto-antioxidant has a TP of 120 to 1200 mg GAE per serving.

In some embodiments, the complex phyto-antioxidant in its natural matrix is from at least one of a fruit, a vegetable, a grain, a seed, a nut, and an herb.

In some embodiments, the food composition further comprises a naturally sourced omega-3 fatty acid.

In some embodiments of the food composition, the vitamin E in its natural matrix is from at least one of grains, wheat germ, vegetable oils, nuts, nut and seed oils, nut butter, sunflower seeds, soybeans, fruit and vegetables.

In some embodiments of the food composition, the vitamin E in its natural matrix has a vitamin E content of 2.7 to 5.4 mg equivalent alpha tocopherol per serving.

In some embodiments, the food composition further comprises at least one stabilizing antioxidant. In particular embodiments, the stabilizing antioxidant comprises vitamin C and/or mixed tocopherols. In particular embodiments, the mixed tocopherols is present in an amount of 5 mg-80 mg per serving. In some embodiments, the expression “mixed tocopherols” refers to an extract from vegetable or palm oils, which consists of a mixture of the different forms of vitamin E including tocotrienols.

In some embodiments, the food composition is provided in a form selected from a bar, a cookie, a cake, a cereal, a liquid product, a semisolid product, a powder, a candy, a snack, a bread, a cracker, a frozen meal, and a frozen confectionary product.

In some embodiments, at least one of the processed ingredients is encapsulated.

In another aspect, a method for preventing or delaying the onset and/or progression of cognitive decline is provided, comprising ingesting a food composition comprising:

a) vitamin E in its natural matrix,

b) a complex phyto-antioxidant in its natural matrix; and

c) at least one processed ingredient present in its synthetic, isolated or extract form.

In some embodiments, vitamin E in its natural matrix is present in an amount of from about 1.4 to about 10.5 mg equivalents alpha tocopherol per serving.

In some embodiments, the complex phyto-antioxidant comprises at least one of an anthocyanin polyphenol, a proanthocyanin polyphenol, a flavanol polyphenol, a flavonol polyphenol, a carotenoid, a terpene and a lignan.

In some embodiments, the cognitive decline is age related or disease related. In particular embodiments, the disease is a neurological disorder.

In some embodiments, the method further comprises cognitive training and/or physical training.

In another aspect, a method for increasing cognitive capacity is provided, comprising ingesting a food composition comprising:

a) vitamin E in its natural matrix,

b) a complex phyto-antioxidant in its natural matrix; and

c) at least one processed ingredient present in its synthetic, isolated or extract form.

In some embodiments, vitamin E in its natural matrix is present in an amount of from about 1.4 to about 10.5 mg equivalents alpha tocopherol per serving.

In some embodiments, the complex phyto-antioxidant comprises at least one of an anthocyanin polyphenol, a proanthocyanin polyphenol, a flavanol polyphenol, a flavonol polyphenol, a carotenoid, a terpene and a lignan.

In some embodiments, the method further comprises cognitive training and/or physical training.

In another aspect, a training regime is provided, comprising:

(a) food composition comprising vitamin E in its natural matrix, a complex phyto-antioxidant in its natural matrix, and at least one processed ingredient present in its synthetic, isolated or extract form; and

(b) a curriculum selected from cognitive training and physical fitness training.

In particular embodiments of the training regime, vitamin E in its natural matrix is provided in an amount of from about 1.4 to about 10.5 mg equivalents alpha tocopherol per serving.

Other features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

In one aspect, the present inventors have invented a delicious convenient source of concentrated brain nutrients. The food composition of this aspect of the present invention is unique in the marketplace comprising functional elements, some of which are in their natural matrix and some of which are in a synthetic, isolate or extract form, such that the food composition is formulated for maximum efficacy. This functionality cannot practically be provided in pills and capsules. In particular, the food compositions of this aspect of the present invention provide certain key nutrients such as vitamin E and phyto-antioxidants in their natural matrix since research has shown that isolation of these nutrients from their natural matrix results in loss of cognitive efficacy. The food composition preferably also comprises other key nutrients as isolates, extracts or synthetics. These processed nutrients have been shown to be as effective or more effective than the corresponding nutrients in their natural matrix.

### I. Definitions

For convenience and clarity certain terms employed in the specification, examples and claims are described herein.

A “functional food” as used herein refers to a food that provides health benefits beyond basic nutrition. Functional foods are regulated by the U.S. Food and Drug Administration (FDA) under the authority of the Federal Food Drug and Cosmetic Act and The Dietary Health Supplements and Education Act (DHSEA).

A “dietary bioactive compound” as used herein refers to a compound having activity that may be isolated from a natural source and includes without limitation, amino acids, vitamins, coenzymes, enzymes, lipids, fatty acids, minerals, complex salts, and biomolecules. The abbreviations “mg” refers to milligrams and “mcg” refers to micrograms.

Quantities of vitamin E may be presented in units of weight, including mg, or in International Units (IU). One mg of vitamin E is equivalent to about 1.5 IU. As is standard in the industry, the vitamin E content is referred to in terms of an equivalent alpha tocopherol content.

As used herein, “natural” or “naturally occurring” refers to ingredients that are extracted directly from plants or animal products as opposed to being produced synthetically.

As used herein, “natural matrix” of a compound refers to the environment of molecules in which it is normally present in the whole food as found in nature except that water may either be present or removed. This ingredient in its natural matrix may then be added to other components to make up the food composition.

As used herein, “processed ingredient” refers to an ingredient which is synthesized or which is extracted or isolated from an edible source as present in nature. As used herein, “natural” or “naturally occurring” refers to processed ingredients that are extracted directly from plants or animal products as opposed to being produced synthetically. For example, fish oils would be a natural processed ingredient whereas dried fish would be in its natural matrix.

As used herein, “cognitive training” includes game playing, computer exercises, reading, puzzles, crossword puzzles, memory games, playing an instrument, chess, bridge, mathematical games and exercises, learning a language, attending classes, staying employed at a job that entails cognitive work, and the like.

As used herein, “physical fitness training” refers to physical activity intended to improve aerobic capacity and increase blood flow and oxygenation to the brain and other vital organs.

### II. Compositions

The generation of reactive oxygen species (ROS) and other free radicals during metabolism is a necessary and normal process that ideally is compensated for by an endogenous antioxidant system. However, in pathological situations excess radicals can accumulate, resulting in oxidative stress. Oxidative stress has been reported to contribute to the etiology of cardiovascular disease, cancer, cell aging and neurodegenerative diseases such as Alzheimer's disease (AD).

Prevention and treatment of this type of damage includes administration of various individual or combinations of anti-oxidants. Antioxidants are compounds that hinder the oxidative processes and thereby delay or prevent oxidative stress.

Accordingly, in one aspect, the present invention provides a food composition comprising:

(a) vitamin E in its natural matrix, the food composition having a vitamin E content of 1.4 to 10.5 mg equivalent alpha tocopherol per serving;

(b) a complex phyto-antioxidant in its natural matrix; and,

(c) at least one processed ingredient that is present in its synthetic, isolated or extract form and that is selected from the group consisting of vitamin B12, vitamin B6, Selenium, Folic acid, Alpha lipoic acid, Ginkgo Biloba, Co-enzyme Q10, Acetyl Carnitine, Phosphatidyl Serine, Vinpocetine, DHA and Epigallocatechin-3-gallate (EGCG), phosphatidyl serine, indole-3-propionic acid, Resveratrol, and salicin from white willow bark.

The vitamin E in its natural matrix has a preferred vitamin E content of 2.7 to 5.4 mg equivalent alpha tocopherol per serving.

Vitamin E actually consists of at least 8 distinct but closely related chemicals divided into two classes, tocopherols and tocotrienols. The first class, tocopherols, have four members designated alpha, beta, gamma and delta. The two major forms a-tocopherol and y-tocopherol, differ structurally only by a methyl group substitution at the 5-position. The second class, tocotrienols, are molecules related to the tocopherols and also consist of four members designated alpha, beta, gamma and delta. The tocotrienol structure differs from the tocopherols by possessing three double bonds in their side chain rather than being saturated. Without wishing to be bound to theory, alpha-tocopherol and these other closely related compounds act as antioxidants. Alpha-tocopherol can prevent lipid peroxidation in vitro, and this function can be replaced by known antioxidants. Alpha-tocopherol also functions as a scavenger of active nitrogen species.

Accordingly, the present food compositions preferably comprise vitamin E in its alpha tocopherol form. In another embodiment, the food composition further comprises at least one additional active form of vitamin E in its natural matrix other than alpha tocopherol, which is preferably the gamma form. The additional active form may be at least one of a tocotrienol, gamma tocopherol and mixed tocopherols. In a preferred embodiment, the tocotrienol is supplied by wheat germ. In another preferred embodiment, the mixed tocopherols are extracted from vegetable or nut oils.

As used herein, the expression “mg equivalents of alpha tocopherol” is a measure of convenience to describe the total amount of active vitamin E forms in a composition. Active vitamin E forms include alpha, beta, delta, and gamma tocopherols, as well as alpha, beta, delta, and gamma tocotrienols. Thus one or more forms of vitamin E may contribute to the total mg equivalents of alpha tocopherol in a composition.

One of the important chemical features of the tocopherols is that they are redox agents, which act under certain circumstances as antioxidants. In acting as an antioxidant tocopherols presumably prevent the formation of toxic oxidation products, such as perioxidation products formed from unsaturated fatty acids. Early on, investigators attributed most if not all of the biological activity of the tocopherols to their ability to act as antioxidants. More recently, however, other biological activities have been associated with tocopherols including the modulation of signal transduction, modulation of phospholipid metabolism, inhibition of protein kinase C, inhibition of phospholipase A and inhibition of prostaglandin production (Meydani and Mosen, 1995).

Further, it has recently been discovered that individual members in the class of tocopherols may exhibit different biological properties from one another despite their structural similarity. Some investigators, for example, believe that γ-tocopherol, unlike α-tocopherol, acts in vivo as a trap for membrane-soluble electrophilic nitrogen oxides and other electrophilic mutagens. (Christen et al., 1997). In contrast, others report that α-tocopherol is a more powerful antioxidant and has ten times the biological activity of γ-tocopherol. (Meydani and Mosen, 1995). Alpha-tocopherol is also thought to be retained in the body longer than γ-tocopherol and has been shown to preferentially reincorporate into nascent very low-density lipoproteins (LDL). (Christen et al., 1997).

The therapeutic benefits of vitamin E supplementation remain a subject of considerable debate. Several studies have proposed that vitamin E supplementation may prevent a plethora of ills but many of these studies fail to provide causal connections between vitamin supplementation and therapeutic benefit; they merely indicate that a high dietary or plasma concentration and supplemental intake of vitamin E is associated with a reduced risk of disease.

In fact, some studies have failed to demonstrate that tocopherol supplementation provides any protection from disease. (Meydani and Mosen, 1995; Christen et al., 1997). Some recent studies indicate that high dose supplemental, as opposed to dietary, vitamin E is associated with some adverse cardiovascular events. A reliable method to treat and prevent diseases associated with oxidative stress and vitamin E deficiency is highly desired.

The vitamin E content, according to the tocopherol content, of certain foods is shown in Table 1 herein below. Values are given in mg/100 g.

TABLE 1
Tocopherol content of representative dietary components

| Dietary Component | α tocopherol | β tocopherol | γ tocopherol |
|---|---|---|---|
| Butter | 1.68 | | 0.14 |
| Lard | 1.20 | | 0.70 |
| Margarine | 3.2-32.70 | | 29 |
| Almonds | 27.40 | 0.30 | 0.90 |
| Sunflower seeds | 49.50 | 2.73 | |
| Cottonseed oil | 38.90 | | 38.7 |
| Corn oil | 11.20 | | 60.20 |
| Sunflower oil | 48.70 | | 5.10 |
| Wheat Germ Oil | 133.00 | 71 | 26 |
| Dehydrated Apricots | 4.33 | | |
| Tomato concentrate | 4.30 | | |
| Pepper, green or red, raw | 1.08 | | |

In one embodiment, the vitamin E in its natural matrix may comprise at least one of grains, wheat germ, vegetable oils, nuts, nut and seed oils, nut butter, sunflower seeds, soybeans and fruits and vegetables. Additional amounts of vitamin E could also be supplied by addition of synthetic vitamin E or vitamin E in extracts of fruits and/or vegetables. The nut may be at least one of walnuts, hazelnuts, almonds, filberts and peanuts. The vegetable may be dried red peppers. The fruit may be dried tomatoes or tomato powder, papaya, mango, blackberries, blueberries, raspberries, strawberries and cranberries.

Almonds and sunflower seeds are one of the primary sources of vitamin E in its natural matrix. Several research studies have found a neuroprotective effect for vitamin E in its natural matrix (Engelhart et al., 2002; Morris et al., 59, 2002) but not when supplied as a supplement. (Petersen et al., 2005). Accordingly, in a particular embodiment, the vitamin E in its natural matrix comprises at least one of sunflower seeds and almonds.

The food compositions of the present invention may also comprise at least one stabilizing antioxidant. In one embodiment, the stabilizing antioxidant is a natural stabilizing antioxidant.

Ascorbic acid reacts readily with oxygen and acts as a reducing agent, thereby serving as an oxygen scavenger. Without wishing to be bound to theory, ascorbic acid can protect vitamin E and other antioxidants in their natural matrix or regenerate phenolic antioxidants by supplying hydrogen atoms to phenoxy radicals that form when the antioxidants yield hydrogen atoms to the lipid oxidation chain reaction. In order to achieve this latter action in lipids, ascorbic acid must be made less polar so it can dissolve in the oil. This may be done by esterification to fatty acids to form a compound such as ascorbyl palmitate.

Accordingly, the natural stabilizing antioxidant may be vitamin C as a water soluble antioxidant protector or mixed tocopherols (as described above), as a fat soluble antioxidant protector. Vitamin C may be present in the food composition in an amount of 50 mg-500 mg per serving, more particularly, 50 mg-350 mg per serving. The mixed tocopherols may be present in an amount of 5 mg-80 mg per serving, more particularly, 16 mg-30 mg per serving. Sources of vitamin C in its natural matrix include tomato, citrus fruit, pomegranate and strawberry. Alternatively, vitamin C may be supplied as a synthesized or purified chemical compound.

The complex phyto-antioxidants of the food composition of the present invention comprise at least one of an anthocyanin polyphenol, a proanthocyanin polyphenol, a flavanol polyphenol, a flavonol polyphenol, a carotenoid, a terpene and a lignan. In one aspect, the complex phyto-antioxidant has a total antioxidant capacity (TAC) of 250 to 5000 micromoles Trolox equivalents (TE) per serving, more preferably, a TAC of 500 to 5000 micromoles TE per serving. In another aspect, the complex phyto-antioxidant has a total phenolic content (TP) of 60 to 1200 mg Gallic Acid Equivalents (GAE) per serving, more preferably, a TP of 120 to 1200 mg GAE per serving. The methodology for the TP and TAC analysis is specified by Xianli Wu et al in the J. Agric. Food Chem. Vol. 52, pp. 4026-4037; 2004.

The complex phyto-antioxidant in its natural matrix of the present invention comprises at least one of a fruit, a vegetable, a grain, a seed, a nut, a herb and a herbal. Additional complex phyto-antioxidant can also be added in an extracted form of fruits and vegetables.

The polyphenols in fruits and vegetables have been shown to be even more powerful antioxidants than vitamins E or C and these antioxidants have been shown to reduce cardiovascular disease which is one of the risk factors for vascular dementia (such as stroke). Accordingly, in another embodiment, the complex phyto-antioxidant in its natural matrix is preferably derived from at least one fruit or at least one vegetable and, more preferably, at least one of (a) at least one fruit and at least one vegetable; (b) at least two fruits and (c) at least two vegetables and, most preferably at least two fruits or two vegetables.

The fruit may be at least one of cranberries, apricots, blueberries, blackberries, prunes, raspberries, strawberries, apples, pomegranates, acai berries, raisins, dates, cherries, red grapes, pineapples, bananas, peaches, plums, pears and figs. In a particular embodiment, the fruit preferably comprises at least one of dried cranberries and dried apricots in an amount of 0.4 g-4.0 g per serving. In a more particular aspect, the fruit preferably comprises at least one of dried cranberries in an amount of 0.8 g-1.5 g per serving and dried apricots in an amount of 1.2 g-2.4 g per serving.

The vegetable may be at least one of red bell peppers, broccoli, brussel sprouts, spinach, kale, red beans, kidney beans and black beans. In a particular embodiment, the vegetable preferably comprises dried red bell peppers in an amount of 0.2 g-4.0 g per serving. In a more particular embodiment, the vegetable preferably comprises dried red bell peppers in an amount of 0.3 g-0.8 g per serving.

The grains and seeds may be at least one of amaranth, barley, buckwheat, corn, flax, kamut, millet, oats, popcorn, quinoa, rice, rye, sesame, spelt, triticale and wheat, preferably at least one of flax, oats and sesame. The herb may be at least one of cinnamon, turmeric, cloves, oregano, parsley, ginger, cloves, rosemary, peppermint, sage, spearmint, savory, thyme and basil. The herb may also be at least one of coffee and tea.

Omega 3 fatty acids have been shown to be important for prevention of cognitive decline (Morris et al., 62, 2005; Giselle et al., 2005). Accordingly, the food compositions of the invention may further comprise a naturally sourced omega-3 fatty acid. In one embodiment, the naturally sourced omega-3 fatty acid is present in the amount of 0.1 g to 4 g per serving, more preferably, 1 g to 3 g per serving. The naturally sourced omega-3 fatty acid may be derived from a plant or a fish or part thereof. In a particular embodiment, the plant comprises at least one of almonds, wheat germ, flaxseed, walnuts and dried algae. In another particular embodiment, the fish or part thereof comprises at least one of salmon, sardine, menhaden and cod liver oil.

Wheat germ is the embryo of the wheat grain and has been shown to contain more nutrients per ounce than any other grain product or vegetable. Its contribution to cognitive enhancement resides in the vitamin E that it contains, its omega 3 fatty acid content and its lecithin content.

Accordingly, in a more particular embodiment, the naturally sourced omega-3 fatty acid preferably comprises at least one of almonds in an amount of 2 g-16 g per serving and wheat germ in an amount of 1.2 g-6 g per serving, more preferably at least one of almonds in an amount of 4 g-8 g per serving and wheat germ in an amount of 2 g-4 g per serving.

Certain nutrients have been shown to be more active in their synthetic, isolated or extract form, such as vitamins B6, B12, folic acid and selenium. High levels of homocysteine have been associated with increased risk of Alzheimers Disease (AD) and AD patients have been found to have low folate levels. vitamins B6, B12, and folate are known to reduce homocysteine levels. Furthermore researchers have-demonstrated the brain protective effects of vitamin B12 (Morris et al., 62, 2005) and folate (Emerson Lombardo et al., 2005). The vitamins are supplied in their synthetic chemical form as these are the forms that have been shown to be active. In fact, in the case of folate the synthetic form is 1.7 times as active as the folate in its natural matrix—probably due to interferences in the natural matrix.

In addition, selenium is a powerful antioxidant in its own right and it tends to act in synergy with vitamin E. The combination of vitamin E and selenium has been shown to have cognitive protection effects in cells and animals. The food compositions of the invention also may have selenium added. This is in addition to the selenium in its natural matrix from wheat germ and other ingredients.

Accordingly, the at least one processed ingredient of the food compositions of the invention preferably comprise at least one of 2 mcg-60 mcg vitamin B12, 0.66 mg-10 mg vitamin B6, 23 mcg-200 mcg Selenium, 50 mcg-1000 mcg Folic acid, 50 mg-600 mg Alpha lipoic acid, 50 mg-250 mg Ginkgo Biloba, 50 mg-1200 mg Co-enzyme Q10, 200 mg-1000 mg Acetyl Carnitine, 50 mg-600 mg Phosphatidyl Serine, 5 mg-60 mg Vinpocetine, 100 mg-1000 mg DHA, 300 mg-2000 mg Epigallocatechin-3-gallate (EGCG), 4-40 mg Resveratrol and 25-80 mg salicin from white willow bark in its synthetic, isolated or extracted form.

In a preferred embodiment of the invention, the at least one processed ingredient preferably comprises at least one of 6 mcg-30 mcg vitamin B12, 2 mg-5 mg vitamin B6, 100 mcg-200 mcg Selenium, 132 mcg-400 mcg Folic acid, 80 mg-200 mg Alpha lipoic acid, 80 mg-200 mg Ginkgo Biloba, 100 mg-300 mg Co-enzyme Q10, 400 mg-800 mg Acetyl Carnitine, 100 mg-300 mg Phosphatidyl Serine, 10 mg-20 mg Vinpocetine, 300 mg-600 mg DHA, 500 mg-1500 mg Epigallocatechin-3-gallate (EGCG), 7-20 mg Resveratrol and 30-60 mg salicin from white willow bark in its synthetic, isolated or extract form.

It is to be understood that certain natural matrices may serve as a source of more than one of the components of the food composition of the present invention, for example, a natural matrix may provide more than one of vitamin E, complex phyto-antioxidants, stabilizing antioxidants and omega-3 fatty acids.

It is also to be understood that the selection of fruit, vegetables, nuts, seeds and or grains and other ingredients may be combined in any permutation that will provide the desired range of vitamin E and complex phyto-antioxidant.

The composition of the invention may be formulated in a variety of products including a bar, a cookie, a cake, a cereal, a liquid product, a semisolid product, a powder, a candy, a snack, a bread, a cracker, a frozen meal and a frozen confectionary product. In a particular embodiment of the present invention, the food composition is provided in a bar. In a more particular embodiment, the compositions may include:

(a) Nut bar, which consists essentially of nuts, seeds and grains. A non-limiting example of a nut bar is a composition consisting essentially of one or more of walnuts, almonds, cashews, peanuts, and hazelnuts; one or more of sunflower seeds and sesame seeds and one or more of wheat, wheat germ, flax, corn, rice, millet and soy.

(b) Fruit and vegetable bar, which consists essentially of fruits and vegetables and nuts and grains. A non-limiting example of a fruit and vegetable bar is a composition consisting essentially of one or more of tomato, apricot, carrot, cranberry, strawberry, apple, grape, citrus fruit and red pepper; one or more of walnuts, almonds, cashews, peanuts, and hazelnuts and one or more of wheat, wheat germ, flax, corn, rice, millet and soy.

(c) Fruit and nut bar, which consists essentially of fruits and nuts and grains. A non-limiting example of a fruit and nut bar is a composition consisting essentially of one or more of tomato, apricot, cranberry, strawberry, apple, grape and red pepper; one or more of walnuts, almonds, cashews, peanuts, and hazelnuts and one or more of wheat, wheat germ, flax, corn, rice, millet and soy. The fruit may be in a dehydrated or liquid form.

(d) Fruit and seed bar, which consists essentially of fruits and vegetables and seeds and grains. A non-limiting example of a fruit and vegetable bar is a composition consisting essentially of one or more of tomato, apricot, carrot, cranberry, strawberry, apple, grape, citrus fruit and red pepper; one or more of sunflower seeds and sesame seeds and one or more of wheat, wheat germ, flax, corn, rice, millet and soy.

The food compositions of the present invention may further comprise a flavor-enhancing ingredient. A person skilled in the art could readily determine the desired flavor-enhancing ingredient. For example, the flavor-enhancing ingredient may be at least one of a natural flavor, an artificial flavor and a sweetener. The food composition may optionally be covered by a flavored coating, for example, chocolate, vanilla, caramel, coconut or sugar coating.

A person skilled in the art would understand that the flavor could be affected by the synthetic, isolated or extracted ingredients. Accordingly, in another embodiment, the processed ingredients are preferably encapsulated in order to maintain a desired flavor.

The food composition of the present invention is preferably made in a serving size that is equal to an amount that is generally consumed by a person in one sitting as a snack. The amount consumed by a person in one sitting as a snack is generally in the range of 100-500 kilocalories.

In a particular embodiment, the serving is about 25 g to 100 g. In a preferred embodiment, the serving is about 30 g to 50 g.

Prevention and treatment of cognitive damage includes administration of various individual or combinations of anti-oxidants. Antioxidants are compounds that hinder the oxidative processes and thereby delay or prevent oxidative stress. Accordingly, the invention provides compositions comprising at least one antioxidant in its natural matrix.

The composition comprises primarily of natural anti-oxidants, vitamins and optionally other nutrients and pharmaceuticals useful for neuroprotection, and delaying onset and treatment of AAMI, MCI, and diseases such as Alzheimer's Disease and Parkinson's Disease and vascular dementia (such as stroke) and others.

Additional naturally occurring compounds such as omega-3 fatty acids, phosphatidyl serine, indole-3-propionic acid, and others that are associated with protection from memory loss and other cognitive losses or promoting memory- or other cognitive functions may be added to the composition.

In some embodiments the composition comprises at least one natural source of an antioxidant selected from vitamin E, vitamin C, vitamin A and derivatives thereof and selenium. In specific embodiments the compositions comprises at least one natural source of vitamin E in its natural matrix.

Vitamin E may be included in both its natural matrix as well as in an extract. In such a case, preferred antioxidants to be included in the compositions include vitamin E (both in its natural matrix and in a processed form) at a range of about 5 mg to about 100 mg per 100 grams. In certain embodiments the composition comprises about 10 mg to about 50 mg per 100 grams. In particular embodiments, the composition comprises about 20 mg to about 40 mg vitamin E per 100 grams food composition.

According to some embodiments, vitamin E is provided as a combination of the four tocopherols and the four tocotrienols. In other embodiments vitamin E includes alpha tocopherol in the preferred form.

Natural sources of vitamin E include grains, wheat germ, nuts including walnuts and almonds, sunflower seed and oils or extracts of these. Other sources include apricots, tomatoes, peppers (preferably red), papaya, mango, blackberries, blueberries, raspberries, strawberries and cranberries and extracts of same.

The composition may further include sources of vitamin C (ascorbic acid) in a range of about 50 mg to about 1000 mg per 100 grams. In some embodiments the composition includes about 50 mg to about 500 mg per 100 grams. Natural sources of vitamin C include fruit such as tomatoes, strawberries and citrus fruit, and extracts thereof.

Other dietary bioactive components include:

Biotin, which may be incorporated at a range of about 30 mcg to about 500 mcg (micrograms) per 100 grams. Biotin may be found in corn, wheat, rice nuts and chocolate.

Folate (vitamin B9) is found in wheat germ and may be included in the composition at a range of about 50 mcg to about 1000 mcg (micrograms) per 100 grams. Good sources of folate include wheat germ, strawberries, blackberries, oranges and red peppers or chemical folic acid.

Vitamin B6, including pyridoxal, pyridoxine, pyridoxamine, and 5′-phosphates (PLP, PMP, PNP), may be included in a range of about 1 mg to about 20 mg per 100 gram, preferably in a range of about 1 mg to about 10 mg per 100 gram. Sources of vitamin B6 include wheat germ, nuts and corn or synthetic sources.

Vitamin B12 (cobalamin) may be included at a range of about 1 mcg to about 60 mcg (microgram), preferably at a range of about 1 mcg to about 10 mcg (microgram). Sources include eggs and dairy products or fermentation source material.

Vitamin A and derivatives including provitamin A and carotenoids are useful antioxidants that may be found in apricots, carrots tomatoes, red pepper and may be included in the composition at a range of about 200-3000 mcg per 100 grams.

Beta-carotene, a vitamin A precursor that is yellow in color and a constituent pigment of green, orange, and yellow vegetables, is one of the most important sources of vitamin A to humans. Beta-carotene yields two molecules of vitamin A per molecule of beta-carotene metabolized, but this conversion is only about 50% efficient in the body. Beta-carotene, as one of the most powerful singlet oxygen quenchers, can dissipate the energy of singlet oxygen ROS, thus preventing this active molecule from generating free radicals. To a lesser extent beta-carotene can also act directly as an antioxidant and scavenge free radicals generated by reactions other than those involving singlet oxygen. Sources of beta-carotene include in a non-limiting matter, yellow, orange or red pepper, pumpkin, orange squash, sweet potatoes, apricot, mango and carrots.

Omega-3 fatty acids are the essential fatty acids known as the “super-unsaturates”. Good sources of omega 3 fatty acids include oils from marine fish, algae, flaxseed, flaxseed oil, canola oil and walnuts and walnut oil. One ounce of walnuts supplies about 2 grams of plant-based omega-3 fatty acids. Soybean oil is also good source but it has omega-6 fatty acids as well. The composition may include about 100 mg to about 10,000 mg omega-3 per 100 grams, and preferably about 200 mg to about 2,000 mg omega-3 per 100 grams.

Selenium is a mineral that functions as an antioxidant. The composition may comprise about 10 mcg to about 1000 mcg per 100 grams, and preferably about 50 mcg to about 500 mcg per 100 grams. Natural sources of selenium include grains and cereals and walnuts.

Other nutrients, which may be included in the composition, include choline (100-2000 mg); niacin (vitamin B3) at about 1 to about 50 mg found for example in rice, corn, nuts and dried fruit; pantothenic acid in a range of about 1 to about 50 mg; riboflavin (vitamin B2) in a range of about 0.1 to about 10 mg; thiamin (vitamin B1) in a range of about 0.1 to about 10 mg; vitamin D (calciferol) at a range of about 1 to about 50 mcg; vitamin K at a range of about 10 to about 500 mcg.

Additional ingredients include ergothianine; phosphatidylserine; phosphorus; zinc; magnesium; copper; manganese; chromium; and molybdenum.

### III. Methods and Uses

The food compositions of the present invention have been developed to provide antioxidants in a natural matrix as well, preferably, as other key nutrients to support and maintain healthy cognitive function. The preferred composition incorporates the requirement to provide vitamin E and complex phyto-antioxidants in their natural matrix with other key nutrients in their synthetic, isolated or extract form in order to maximize the efficacy of these nutrients.

Accordingly, in another aspect, the invention provides the use of a food composition comprising at least one antioxidant in its natural matrix for preventing or delaying at least one of the onset and the progression of cognitive decline. The invention also provides a method of preventing or delaying at least one of the onset and the progression of cognitive decline in a human subject comprising administering at least one antioxidant in its natural matrix in a sufficient amount useful for preventing or delaying at least one of the onset and the progression of cognitive decline.

The human may be any age range. In a preferred embodiment, the human is a person over age of 40 years. The food composition may be used or administered before the onset and progression of cognitive decline or after the onset. The food compositions may be a supplement for people taking prescription medicines.

In another embodiment, the invention provides the use of one of the food compositions for preventing or delaying at least one of the onset and the progression of cognitive decline. The invention also provides a method of preventing or delaying at least one of the onset and the progression of cognitive decline in a human subject comprising administering one of the food compositions of the invention in a sufficient amount useful for preventing or delaying at least one of the onset and the progression of cognitive decline.

In one embodiment, the cognitive decline is age related or disease related. In a particular embodiment, cognitive decline is Age-Associated Memory Impairment (AAMI) or Mild Cognitive Impairment (MCI). In another particular embodiment, the disease related cognitive decline is associated with neurodegenerative disease. The neurodegenerative disease may be Alzheimer's Disease or Parkinson's Disease, or vascular dementia (such as stroke).

In another aspect, the invention provides the use of a food composition comprising at least one antioxidant in its natural matrix for increasing cognitive capacity. The invention also provides a method for increasing cognitive capacity in a human subject comprising administering a food composition comprising at least one antioxidant in its natural matrix in a sufficient amount suitable for increasing cognitive capacity.

In another embodiment, at least one antioxidant of the uses and methods of the invention comprises at least one of vitamin E and a complex phyto-antioxidant in its natural matrix. In a preferred embodiment, the vitamin E in its natural matrix has a vitamin E content of 1.4 to 10.5 mg equivalent alpha tocopherol per serving wherein at least one serving is consumed per week, and preferably per day In a preferred embodiment, the vitamin E in its natural matrix has a vitamin E content of 2.7 to 5.4 mg equivalent alpha tocopherol per serving wherein at least one serving is consumed per week, and preferably per day.

Both cognitive training and physical activity have been shown to be beneficial to overall well being of adults and the elderly. Numerous research studies have shown that older adults benefit from formal training programs aimed at specific cognitive abilities. Late life intellectual stimulation may reduce or delay onset of age related and disease related dementia. Sustained engagement in cognitively stimulating activities has been found to positively impact neural structure in the elderly, resulting in improved memory function, faster reaction and processing speeds and improved reasoning and problem solving capabilities.

A positive relationship between regular participation in physical fitness and a lower risk of acquiring Alzheimer's Disease has been reported (Canadian Study of Health and Aging, 2002). Examples of physical fitness include walking, yoga, calisthenics, flexibility training, movement therapy, Feldenkreis training, bicycling, swimming and/or aerobic water-based exercises, Pilates, gymnastics, folk dancing, Alexander technique, Tai Chi and similar.

Accordingly, in another embodiment, the uses and methods of the invention further comprise a curriculum, the curriculum comprising at least one of cognitive training and physical fitness training. In one embodiment, the curriculum comprises a combination of cognitive training and physical training.

The cognitive training of the curriculum may be for about 30 minutes to about 14 hours per week, preferably for about 5 minutes to about 2 hours per day and more preferably for about 15 minutes to about 60 minutes per day.

The physical fitness training of the curriculum may be for about 30 minutes to about 14 hours per week, preferably for about 5 minutes to about 120 minutes per day and more preferably for about 15 minutes to about 60 minutes per day.

Cognitive training may be administered to a subject via exercises such as are found in software products, paper exercises or any cognitive activity designed to improve the symptoms associated with cognitive decline including loss of memory, speed of processing, learning, etc. The cognitive training may be administered from about 1-7 days per week, preferably from about 3 to about 7 days per week. The cognitive training may be administered once a day or at various intervals through the day. The physical fitness may be administered from about 1-7 days per week, preferably from about 3 to about 7 days per week. The cognitive training may be administered once a day or at various intervals through the day.

In some embodiments, the curriculum selected from cognitive training and physical fitness training is self-administered. In other embodiments, a fitness professional may offer the curriculum.

### IV. Training Regimes

In yet another aspect, the invention provides a training regime comprising

(a) consuming a food composition comprising at least one antioxidant in its natural matrix; and

(b) a curriculum selected from cognitive training and physical fitness training.

In one embodiment, the at least one antioxidant comprises at least one of vitamin E and a complex phyto-antioxidant in its natural matrix. In a particular embodiment, the vitamin E in its natural matrix has a vitamin E content of 1.4 to 10.5 mg equivalent alpha tocopherol per serving wherein at least one serving is consumed per week, and preferably per day. In a preferred embodiment, the vitamin E in its natural matrix has a vitamin E content of 2.7 to 5.4 mg equivalent alpha tocopherol per serving wherein at least one serving is consumed per week, and preferably per day.

In another embodiment, the invention provides a training regime comprising

(a) consuming a food composition of the present invention; and

(b) a curriculum selected from cognitive training and physical fitness training.

In one embodiment, the curriculum of the training regime comprises a combination of cognitive training and physical training. The cognitive training of the curriculum may be for about 30 minutes to about 14 hours per week, preferably for about 5 minutes to about 2 hours per day and more preferably for about 15 minutes to about 60 minutes per day.

The physical fitness training of the curriculum may be for about 30 minutes to about 14 hours per week, preferably for about 5 minutes to about 120 minutes per day and more preferably for about 15 minutes to about 60 minutes per day.

Cognitive training may be administered to a subject via exercises such as are found in software products, paper exercises or any cognitive activity designed to improve the symptoms associated with cognitive decline including loss of memory, speed of processing, learning, etc. The cognitive training may be administered from about 1-7 days per week, preferably from about 3 to about 7 days per week. The cognitive training may be administered once a day or at various intervals through the day. The physical fitness may be administered from about 1-7 days per week, preferably from about 3 to about 7 days per week. The cognitive training may be administered once a day or at various intervals through the day.

In some embodiments, the curriculum selected from cognitive training and physical fitness training is self-administered. In other embodiments, a fitness professional may offer the curriculum.

The above disclosure generally describes the present invention. A more complete understanding can be obtained by reference to the following specific examples. These examples are described solely for the purpose of illustration and are not intended to limit the scope of the invention. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present invention:

### EXAMPLES

### Example 1

### Formulations of Fruit and Vegetable Bars

The compositions detailed in Table 2 were prepared by mixing the moist ingredients together, and adding the dry ingredients to the moist mixture. The entire mixture was then laminated and cut into bars. The bars were tested for organoleptic properties and packaged for testing shelf life.

TABLE 2
Composition of certain embodiments of the invention

| Raw Material | 1 | 2 | 3 | 4 | 5 | 6 |
|---|---|---|---|---|---|---|
| Moist ingredients: | | | | | | |
| Fructose | 480 | 480 | | 210 | 270 | 210 |
| Apple sauce | 120 | 120 | 240 | 100 | 100 | 100 |
| Dehydrated apricot | 120 | 120 | 80 | 210 | 210 | 210 |
| Maltidex | | | 360 | | | |
| Water | | | | | 60 | |
| Dehydrated Cranberries | 20 | 70 | | 60 | 60 | 60 |
| Citric Acid (25% sol) | 0 | 5 | 20 | 22 | 22 | 22 |
| Apricot flavor | | 0.25 | 1 | 2 | 2 | 2 |
| Total | 740 | 795 | 701 | 604 | 724 | 604 |
| Remove and add to | 520 | 520 | 500 | 520 | 520 | 520 |
| Dry ingredients: | | | | | | |
| Sunflower seeds | 140 | 140 | 140 | 200 | 200 | 200 |
| Natural almonds | 140 | 140 | 140 | | 200 | 100 |
| Roasted Almonds | | | | 200 | | 100 |
| Wheat Germ | 170 | 170 | 90 | 70 | 0 | 0 |
| Wheat Crisps | 170 | 170 | 120 | 120 | 60 | 50 |
| Rice Crisps | | | | | 60 | 70 |
| Dehydrated pepper | 20 | 20 | 40 | 20 | 20 | 20 |
| Total weight | 1160 | 1160 | 1080 | 1130 | 1060 | 1060 |

The unit of weight for the following table is grams. The approximate nutritional values for the above products above are shown in Table 3, herein below:

TABLE 3
Approximate nutritional values for the above products

| | Per | Per 1 bar | Coated - | Coated - |
|---|---|---|---|---|
| Nutritional Value | 100 g | (25 g) | (100 g) | (35 g) |
| Energy (Kcal) | 412 | 105 | 445 | 155 |
| Fat (g) | 16.5 | 4 | 20 | 5 |
| Sat. Fat | 1.5 | 0.45 | 5 | 2 |
| Sodium (mg) | 10 | 2.5 | 12 | 3 |
| Carbohydrates (g) | 56.5 | 14 | 58 | 14.5 |
| Sugars (g) | 35 | 9 | 40 | 10 |
| Dietary Fiber (g) | 2.2 | 0.6 | 2 | 0.5 |
| Protein (g) | 10 | 2.5 | 9 | 2 |
| Vitamin E | 26 | 6.5 | 21 | 7 |

Some of the compositions were coated with a milk chocolate or dark chocolate coating. Compositions 4 and 5 proved to be the tastiest and have the best texture. Composition 3 tasted good but was too soft for a bar. After two weeks in storage the bars were tested for shelf life. Some of the bars had lost their crispness. In some embodiments a soft, chewy bar is desirable.

### Example 2

### Fruit and Seed and Nut Bars

Compositions of fruit and seed and nut bars were prepared and some of these are shown in Table 4. The nutritional values for an exemplary bar, derived from the composition in Table 4, are shown in Table 5.

TABLE 4
Compositions of fruit and seed and nut bars

| Raw Material | 7 | 8 | 9 |
|---|---|---|---|
| Moist Ingredients | | | |
| Sugar (g) | 450 | 450 | 450 |
| Water ml) | 165 | 165 | 165 |
| Corn syrup | 85 | 85 | 85 |
| Citric Acid (10% sol) | 4.5 | 4.5 | 4.5 |
| Total | 704.5 | 704.5 | 704.5 |
| Remove and add to | 255 | 280 | 280 |
| Dry ingredients: | | | |
| Sunflower seeds | 135 | 135 | 135 |
| Natural Almonds | 102 | 68 | 68 |
| Dehydrated Apricots | 55 | 55 | 55 |
| Dehydrated Cranberries | 24 | 24 | 24 |
| Roasted Almonds | 34 | 68 | 68 |
| Wheat Germ | 68 | 68 | 68 |
| Wheat Crisps | 34 | 34 | 34 |
| Rice Crisps | 48 | 48 | 48 |
| Nat. antioxidant | 1 | | 1 |
| Dehydrated pepper | 14 | 14 | 14 |
| Apricot flavor | 1 | 1 | 1 |
| Citric acid (10% solu) | 5 | 5 | 10 |
| Total weight | 776 | 800 | 806 |

TABLE 5
Nutritional values of exemplary bars

| | Per | Per 1 bar | Coated - | Coated - |
|---|---|---|---|---|
| Nutritional Value | 100 g | (25 g) | 100 g | 35 g |
| Energy (Kcal) | 425 | 106 | 457 | 160 |
| Fat (g) | 18.6 | 4.5 | 21 | 7 |
| Sat. Fat (g) | 1.7 | 0.4 | 6 | 2 |
| Sodium (mg) | 12 | 3 | 14 | 5 |
| Carbohydrates (g) | 54 | 14 | 59 | 21 |
| Sugars (g) | 35 | 9 | 40 | 10 |
| Dietary Fiber (g) | 2.2 | 0.6 | 2 | 0.5 |
| Protein (g) | 10 | 2.5 | 9 | 2 |
| Vitamin E | 26 | 6.5 | 21 | 7 |

### Example 3

### Cognitive Bar Composition

The following is a summary of the compositional aspects of a cognitive bar as presented below. The summary is divided into (a) those components that are important to be supplied in their natural matrix and (b) those components that are more efficaciously supplied as isolated or synthesized compounds.

TABLE 6
Components in Their Natural Matrix

| | | Amount | Amount | |
|---|---|---|---|---|
| | | per Bar - | per Bar - | |
| Active | Source | Range | Preferred | Alternatives |
| Vitamin E | Sunflower | 2 g-16 g | 4 g-8 g | Hazelnuts, |
| (various | seeds¹ | | | filberts, |
| forms) | | | | peanuts |
| | Almonds¹ | 2 g-16 g | 4 g-8 g | As above |
| | Wheat germ² | 1.2 g-6 g | 2 g-4 g | |
| | Mixed | 5 mg-80 mg | 16 mg-30 mg | |
| | tocopherols³ | | | |
| Complex | Dried | 0.4 g-4.0 g | 0.8-1.5 g | Other coloured |
| phyto- | Cranberries | | | fruits & fruit |
| anti- | | | | juices (apple, |
| oxidants⁴ | | | | grape) & |
| | | | | vegetables |
| | Dried Red | 0.2 g-4.0 g | 0.3 g-0.8 g | As above |
| | bell peppers | | | |
| | Apricots | 0.4 g-4.0 g | 1.2 g-2.4 g | As above |
| Omega-3 | Almonds | See above | See above | Fish oil, |
| fatty | | | | Docosa- |
| acids⁵ | | | | hexaenoic |
| | | | | acid (DHA), |
| | | | | flax seeds, |
| | | | | walnuts, |
| | | | | purslane |
| | Wheat germ | See above | See above | As above |

¹Highest nut sources of natural vitamin E and among highest food sources.
²Also contains other nutrients such as selenium, omega-3 fatty acids etc.
³Excellent source of Vit E forms other than alpha tocopherol and protection for other sources.
⁴Tend to be ill defined. Ingredients chosen contain phyto-antioxidants but also vitamin E and other actives.
⁵Almonds & wheat germ supply alpha linolenic acid form of omega-3

TABLE 7
Components as Isolates/Extracts or Synthetics

| | | Amount per Bar - | Amount per Bar - |
|---|---|---|---|
| Active | Source | Range | Preferred |
| Vitamin B12 | Chemical | 2 mcg-60 mcg | 6 mcg-30 mcg |
| Folic Acid | Chemical | 50 mcg-1000 mcg | 132 mcg-400 mcg |
| Vitamin B6 | Chemical | 0.66 mg-10 mg | 2 mg-5 mg |
| Selenium | Chemical | 23 mcg-200 mcg | 100 mcg-200 mcg |
| Vitamin C | Chemical | 50 mg-500 mg | 50 mg-350 mg |
| Alpha lipoic acid | Chemical | 50 mg-600 mg | 80 mg-200 mg |
| Ginkgo Biloba | Extract | 50 mg-250 mg | 80 mg-200 mg |
| Co-enzyme Q10 | Chemical | 50 mg-1200 mg | 100 mg-300 mg |
| Acetyl Carnitine | Chemical | 200 mg-1000 mg | 400 mg-800 mg |
| Phosphatidyl | Chemical/ | 50 mg-600 mg | 100 mg-300 mg |
| Serine | Extract | | |
| Vinpocetine | Extract | 5 mg-60 mg | 10 mg-20 mg |
| DHA | Extract | 100 mg-1000 mg | 300 mg-600 mg |
| Epigallocatechin- | Extract | 300 mg-2000 mg | 500 mg-1500 mg |
| 3-gallate (EGCG) | | | |

### Example 4

### Coated Food Bar

| | Wt g/lab | Wt./39 g | |
|---|---|---|---|
| Ingredient | batch | bar | % |
| Chocolate Coating | 99.53 | 5.85 | 15.00% |
| Honey | 60.35 | 3.55 | 9.10% |
| Brown Rice Syrup | 84.60 | 4.97 | 12.75% |
| Sunflower seeds | 108.85 | 6.40 | 16.41% |
| Prehyd. Xanthan Gum | 0.45 | 0.03 | 0.07% |
| Dehydrated apricot pieces | 44.27 | 2.60 | 6.67% |
| Dried cranberry pieces | 19.18 | 1.13 | 2.89% |
| Roasted almond pieces | 108.85 | 6.40 | 16.41% |
| Wheat germ | 54.42 | 3.20 | 8.20% |
| Soy crisps | 27.18 | 1.60 | 4.10% |
| Rice crisps | 33.84 | 1.99 | 5.10% |
| Dehydrated red ball peppers | 11.22 | 0.66 | 1.69% |
| Citric acid | 3.38 | 0.20 | 0.51% |
| Apricot flavor | 1.24 | 0.07 | 0.19% |
| Mixed tocopherols | 1.26 | 0.22 | 0.19% |
| Vitamin mix | 4.91 | 0.29 | 0.74% |
| TOTAL | 663.52 | 39.15 | 100.00% |

The vitamin mix contains B6, B12, C, Folate—All encapsulated and vitamin E. Also contains Pantothenic, Riboflavin, Thiamin, Vit. A, Niacin, zinc, magnesium, copper—all encapsulated and biotin, selenium, chromium—not encapsulated.

The mixed tocopherols are diluted 1 part mixed tocopherols to 4 parts soya oil.

Procedure—Add mixed tocopherols (1 part) to soya oil (4 parts) at 25-60 deg C. and mix well. Add citric acid, mixed tocopherols preblend, and flavor to binding syrup, mix well and then add remaining ingredients & mix before extrusion.

### Example 5

### Uncoated Food Bar

| | Wt g/lab | Wt./33 g | |
|---|---|---|---|
| Ingredient | batch | bar | % |
| Honey | 70.99 | 3.53 | 10.70% |
| Brown Rice Syrup | 99.53 | 4.95 | 15.00% |
| Sunflower seeds | 128.06 | 6.37 | 19.30% |
| Prehyd. Xanthan Gum | 0.53 | 0.03 | 0.08% |
| Dehydrated apricot pieces | 52.08 | 2.59 | 7.85% |
| Dried cranberry pieces | 22.56 | 1.12 | 3.40% |
| Roasted almond pieces | 128.06 | 6.37 | 19.30% |
| Wheat germ | 64.03 | 3.18 | 9.65% |
| Soy crisps | 31.98 | 1.59 | 4.82% |
| Rice crisps | 39.81 | 1.98 | 6.00% |
| Dehydrated red ball peppers | 13.20 | 0.66 | 1.99% |
| Citric acid | 3.98 | 0.20 | 0.60% |
| Apricot flavor | 1.46 | 0.07 | 0.22% |
| Mixed tocopherols | 1.46 | 0.22 | 0.22% |
| Vitamin mix | 5.77 | 0.29 | 0.87% |
| TOTAL | 663.5 | 33.15 | 100.00% |

The vitamin mix contains B6, B12, C, Folate—All encapsulated and vitamin E. Also contains Pantothenic, Riboflavin, Thiamin, Vit. A, Niacin, zinc, magnesium, copper—all encapsulated and biotin, selenium, chromium—not encapsulated.

The mixed tocopherols are diluted 1 part mixed tocopherols to 4 parts soya oil.

Procedure—Add mixed tocopherols (1 part) to soya oil (4 parts) at 25-60 deg C. & mix well. Add citric acid, mixed tocopherols preblend, and flavor to binding syrup, mix well and then add remaining ingredients & mix before extrusion.

### Example 6

### Physical Activity and Cognitive Exercise Programs

A curriculum for physical activity may be prepared on an individual basis for individual or group activity. The activities are selected to suit an individual's interests, health and mobility limitations, and time, financial and climate considerations. Likewise a cognitive training curriculum may be selected according to an individual's interests and needs.

According to certain embodiments, an individual is engaged in fitness and or cognitive activities for about 30 minutes to about 14 hours per week. The activities are preferably distributed throughout the week, over three to seven days. According to some embodiments the activities are performed on a daily basis.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1-116. (canceled)

117. A food composition comprising:
a) Vitamin E in its natural matrix, in an amount of from about 1.4 to 10.5 mg equivalents alpha tocopherol per serving;
b) a complex phyto-antioxidant in its natural matrix; and
c) at least one processed ingredient present in its synthetic, isolated or extract form and selected from the group consisting of Vitamin B12, Vitamin B6, Selenium, Folic acid, Alpha lipoic acid, Ginkgo Biloba, Co-enzyme Q10, Acetyl Carnitine, Phosphatidyl Serine, Vinpocetine, DHA, Epigallocatechin-3-gallate (EGCG), phosphatidyl serine, indole-3-propionic acid, Resveratrol, and salicin from white willow bark.

118. The food composition of claim 117, wherein the complex phyto-antioxidant comprises at least one of an anthocyanin polyphenol, a proanthocyanin polyphenol, a flavanol polyphenol, a carotenoid, a terpene and a lignan.

119. The food composition of claim 117, wherein the complex phyto-antioxidant has a total antioxidant capacity (TAC) of 250 to 5000 micromoles Trolox equivalents (TE) per serving.

120. The food composition of claim 117, wherein the complex phyto-antioxidant has a total phenolic content (TP) of 60 to 1200 mg Gallic Acid Equivalents (GAE) per serving.

121. The food composition of claim 117, wherein the complex phyto-antioxidant in its natural matrix is from at least one of a fruit, a vegetable, a grain, a seed, a nut, and an herb.

122. The food composition of claim 117, further comprising a naturally sourced omega-3 fatty acid.

123. The food composition according to claim 117, wherein the vitamin E in its natural matrix is from at least one of grains, wheat germ, vegetable oils, nuts, nut and seed oils, nut butter, sunflower seeds, soybeans, fruit and vegetables.

124. The food composition according to claim 117, wherein the vitamin E in its natural matrix has a vitamin E content of 2.7 to 5.4 mg equivalent alpha tocopherol per serving.

125. The food composition of claim 117, further comprising at least one stabilizing antioxidant.

126. The food composition of claim 125, wherein the stabilizing antioxidant comprises vitamin C or mixed tocopherols.

127. The food composition of claim 126, wherein the mixed tocopherols is present in an amount of 5 mg-80 mg per serving.

128. The food composition of claim 117, wherein the food composition is provided in a form selected from a bar, a cookie, a cake, a cereal, a liquid product, a semisolid product, a powder, a candy, a snack, a bread, a cracker, a frozen meal, and a frozen confectionary product.

129. The food composition according to claim 117, wherein at least one of the processed ingredients is encapsulated.

130. A method for preventing or delaying the onset and/or progression of cognitive decline, comprising ingesting a food composition comprising:
a) vitamin E in its natural matrix,
b) a complex phyto-antioxidant in its natural matrix; and
c) at least one processed ingredient present in its synthetic, isolated or extract form.

131. The method of claim 130, wherein vitamin E in its natural matrix is in an amount of from about 1.4 to about 10.5 mg equivalents alpha tocopherol per serving.

132. The method of claim 130, wherein the complex phyto-antioxidant comprises at least one of an anthocyanin polyphenol, a proanthocyanin polyphenol, a flavanol polyphenol, a carotenoid, a terpene and a lignan.

133. The method of claim 130, wherein the cognitive decline is age related or disease related.

134. The method of claim 133, wherein the disease is a neurological disorder.

135. The method of claim 130, further comprising cognitive training or physical training.

136. A method for increasing cognitive capacity, comprising ingesting a food composition comprising:
a) vitamin E in its natural matrix,
b) a complex phyto-antioxidant in its natural matrix; and
c) at least one processed ingredient present in its synthetic, isolated or extract form.

137. The method of claim 136, wherein vitamin E in its natural matrix is in an amount of from about 1.4 to about 10.5 mg equivalents alpha tocopherol per serving.

138. The method of claim 136, wherein the complex phyto-antioxidant comprises at least one of an anthocyanin polyphenol, a proanthocyanin polyphenol, a flavanol polyphenol, a carotenoid, a terpene and a lignan.

139. The method of claim 136, further comprising cognitive training or physical training.

140. A training regime comprising:
(a) food composition comprising vitamin E in its natural matrix, a complex phyto-antioxidant in its natural matrix, and at least one processed ingredient present in its synthetic, isolated or extract form; and
(b) a curriculum selected from cognitive training and physical fitness training.

141. The training regime of claim 140, wherein vitamin E in its natural matrix is provided in an amount of from about 1.4 to about 10.5 mg equivalents alpha tocopherol per serving.
